(19) United States
(12) Patent Application Publication
(10) Pub. No.: US 2002/0177136 A1
(43) Pub. Date: Nov. 28, 2002

(54) Peptide nucleic acid based molecular sensors for nucleic acids

(76) Inventors: Duncan W. McBranch, Santa Fe, NM (US); David G. Whitten, Santa Fe, NM (US); Michael Egholm, Woodbridge, CT (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 09/934,680

(22) Filed: Aug. 23, 2001

Related U.S. Application Data
(60) Provisional application No. 60/226,902, filed on Aug. 23, 2000.

(51) Int. Cl. C12Q 1/68; C07H 21/04

(52) U.S. Cl. 435/6; 534/727; 536/24.3

(57) ABSTRACT

A chemical moiety including a polymer (P), a first tethering element (T), a ligand (L) which is a specific sequence of PNA, a second tethering element (T′) and a quencher (Q) is disclosed. In the absence of a complement to the PNA sequence, the PNA is in a tightly coiled configuration, thereby quenching the polymer due to the close proximity of the quencher to the polymer. When a receptor is added that recognizes the PNA sequence, a hybridization of the PNA sequence separates the polymer and the quencher, resulting in an increase of detected fluorescence. The same chemistry is advantageously employed in a competitive assay. A method for detecting nucleic acids in a target sample using the PTLT′Q molecule is also disclosed.

This application claims priority from U.S. Provisional Application Serial No. 60/226,902 filed Aug. 23, 2000. The entirety of that provisional application is incorporated herein by reference.

### BACKGROUND OF THE INVENTION

1. Field of the Invention

The present invention relates to a fluorescent biosensor that functions by a novel Quencher-Tether-Ligand mechanism. In particular, a Polymer-Tether-Ligand-Tether-Quencher (PTLT′Q) system provides for effective sensing of nucleic acids by observing fluorescence changes.

2. Discussion of the Background

Sequence specific recognition of DNA or RNA provides the most flexible and effective strategy for the detection and identification of many types of biological agents, such as biowarfare agents (e.g., anthrax). Of the many strategies developed for molecular recognition of nucleic acids, the most powerful remains hybridization. Hybridization is a straightforward strategy for the detection of DNA. (1).

In hybridization, a nucleic acid or synthetic analogue reads the sequence information on the target nucleic acid and then forms a double-helical complex with the target molecule through complementary Watson-Crick base pairing. Typically, a 10-20 nucleotide subsequence from the target nucleic acid is selected and a complementary probe sequence is synthesized. Alternatively, a gene probe several hundred base pairs in length can be synthesized using polymerase chain reaction (PCR). Polymerase chain reaction (PCR) is an in vitro method for amplifying specific DNA sequences using a heat-stable polymerase and two 20-base primers. One primer is complementary to the (+)-strand at one end of the sequence to be amplified and the other primer is complementary to the (−)-strand at the other end. Because the newly synthesized DNA strands can subsequently serve as additional templates for the same primer sequences, successive rounds of primer annealing, strand elongation, and dissociation produce rapid and highly specific amplification of the desired sequence. Once the gene probe is synthesized, the target DNA is added and hybridization is allowed to proceed. Successful hybridization can be detected by autoradiography, calorimetric analysis, or chemiluminescence.

Hybridization approaches are extremely versatile since alteration of the assay to detect mutated forms of the original target requires only a redesign of the probe sequence to regain full complementarity. Furthermore, hybridization allows a high level of control over recognition and permits the ability to discriminate between target sequences that differ at only a single position.

However, the use of DNA in hybridization techniques has several drawbacks. For example, complementary DNA strands must overcome electrostatic repulsions in order to hybridize with one another. This effectively makes the reaction less thermodynamically favorable than if the probe strand were uncharged or cationic. Additionally, single-stranded DNA is susceptible to degradation by exonuclease enzymes. Further, the use of DNA requires that the samples be rigorously purified, thereby adding time to complete the assay.

The ability to rapidly and accurately detect and quantify nucleic acid molecules with high sensitivity is a central issue for medical technology, national security, public safety, and civilian and military medical diagnostics. Although DNA hybridization offers a sensitive and straightforward method of detecting DNA, the use of DNA has several drawbacks which make it a less than ideal method. In order to address the problems in the art, the present inventors have developed a prototype for a new fluorescent biosensor which functions by a novel Polymer-Tether-Ligand-Tether-Quencher (PTLT′Q) mechanism that provides for simple, rapid and highly-sensitive detection of nucleic acids.

### SUMMARY OF THE INVENTION

It is an object of the invention to provide a novel chemical moiety formed of a polymer (P), a first tethering element (T), a ligand (L), a second tethering element (T′), and a quencher (Q).

It is another object of the present invention to provide a method of detecting nucleic acids in a target sample using the novel PTLT′Q molecule of the present invention.

It is further object of the present invention to detect target nucleic acids in a sample by observing fluorescent changes.

It is a feature of the present invention that the change in fluorescence is indicative of the presence of the target nucleic acid.

It is an object of the present invention to rapidly and accurately detect target nucleic acids in a sample.

It is an advantage of the present invention that it is simple and requires no elaborate preprocessing.

These and other objects of the present invention are met by a composition of matter comprising a chemical moiety including a fluorescent polymer bound together by a first tethering element to a recognition element which binds to a target nucleic acid. The recognition element is further bound together by a second tethering element to a property altering element which alters fluorescence emitted by the fluorescent polymer when complexed together to a distinguishable degree. In the presence of binding of the recognition element to the target nucleic acid, the fluorescence emitted by said fluorescent polymer is altered from that emitted when said binding between the recognition element and the target nucleic acid does not occur.

### BRIEF DESCRIPTION OF THE DRAWINGS

FIG. 1 is a general illustration of a fluorescence “turn-on” assay using a fluorescent polymer.

FIG. 2 is a general illustration of a competitive assay using a QTL bioconjugate.

FIG. 3 is an illustration comparing DNA and PNA chemical structures.

FIGS. 4A-4C are illustrations of PNA strand invasion.

FIG. 5 is a general illustration of the PTLT′Q molecule.

FIG. 6 is a general illustration of a competitive assay using the PTLT′Q molecule.

### DESCRIPTION OF THE PREFERRED EMBODIMENTS

The key scientific basis for the polymer QTL approach is the amplification of quenching of fluorescence that may be obtained with certain charged polymers, including, but not limited to, conjugated polymers and dye-pendant polyelectrolytes in which the chromophores are collected by non-covalent interactions (e.g., J-aggregation), and small molecule quenchers. The fluorescent polymers provide amplification over conventional molecular fluorophores both by virtue of their light-harvesting properties (collective excitation) and their sensitivity to superquenching (i.e., one quencher may extinguish luminescence from an entire polymer chain).

Fluorescent polymer superquenching has been adapted to biosensing applications through the use of “QTL” bioconjugates. (2-7). QTL bioconjugates contain a small molecule electron transfer or energy transfer quencher (Q), linked through a tether (T) to a ligand (L) for a specific bioagent.

Suitable examples of ligands that can be used in “QTL” methods include chemical ligands, hormones, antibodies, antibody fragments, oligonucleotides, antigens, polypeptides, glycolipids, proteins, protein fragments, enzymes, peptide nucleic acids (PNAs), and polysaccharides. Examples of suitable tethers include, without limitation, single bonds, single divalent atoms, divalent chemical moieties of up to approximately 10 carbon atoms in length, multivalent chemical moieties, polyethylene, polyethylene oxides, polyamides, non-polymeric organic structures of at least about 7-20 carbon atoms, and related materials. Suitable quenchers include methyl viologen, quinones, metal complexes, fluorescent and nonfluorescent dyes, and energy accepting, electron accepting, and electron donating moieties. However, these examples of the ligand, tethering elements, and quenchers are not to be construed as limiting, as other suitable examples would be easily determined by one of skill in the art.

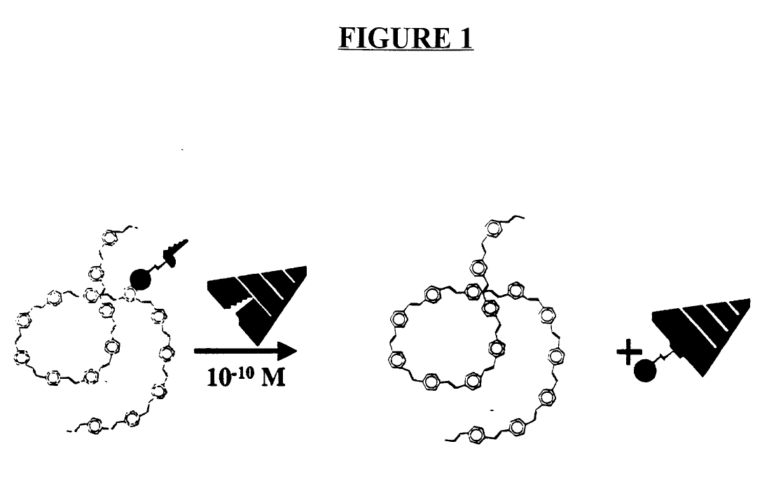

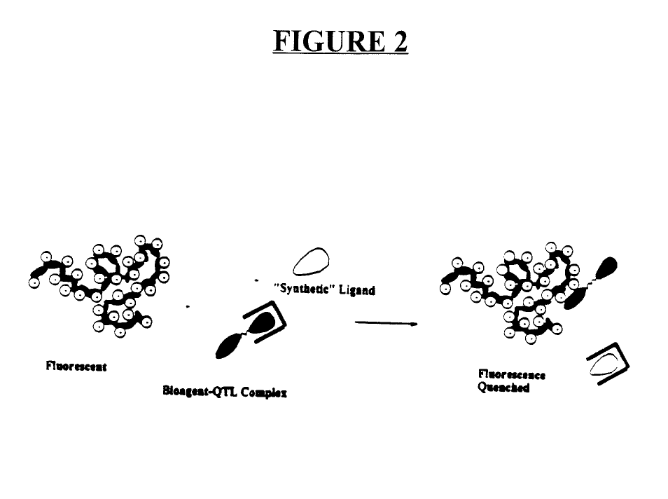

In its simplest example, a small amount of QTL bioconjugate is added to a solution of a fluorescent polyelectrolyte which results in a quenching of the polymer fluorescence. As shown in FIG. 1, the addition of a target bioagent recognizing the ligand L results in a removal of the QTL bioconjugate from the polymer and an increase or “turn-on” of fluorescence. (4). In addition, it has been shown that the QTL bioconjugate superquenching can be used in a competitive assay. In this case, the QTL bioconjugate is precomplexed with the bioagent and the polymer fluorescence is initially unquenched as shown in FIG. 2. Addition of a natural or synthetic ligand results in competition for the binding site of the bioagent, release of the QTL bioconjugate, and quenching of the polymer fluorescence.

The present invention is a further extension of the use of superquenching for biosensing. In particular, a sensor including a fluorescent polymer (P) (or oligomer) and a quencher (Q) have been linked intramolecularly by a tether containing a segment of peptide nucleic acids (PNA) that can recognize and hybridize with a specific sequence of nucleic acid, such as either DNA or RNA.

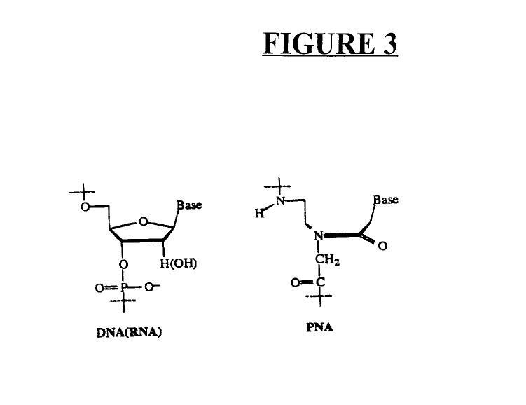

PNA was first described in 1991 by Buchardt, Neilsen, Egholm, and Berg at the University of Copenhagen. (8). As can be seen in FIG. 3, PNA maintains the same number of single bonds in the backbone and separating the hydrogen bonding nucleobase from the backbone as is found in DNA and RNA. However, the PNA backbone is uncharged, thereby permitting much higher affinity hybridization with complementary DNA and RNA strands in comparison with analogous DNA probes. (9). In addition, the unnatural structure of PNA renders it extremely stable to both protease and nuclease enzymes. (10).

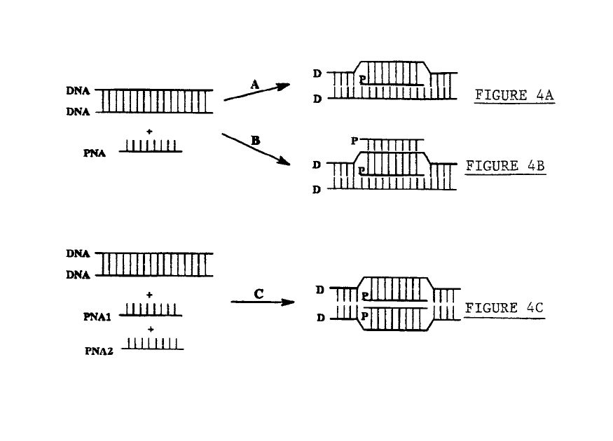

A significant advantage offered by PNA relative to DNA-based probes is the ability of PNA to bind double-stranded DNA targets. In the vast majority of cases, genomic DNA exists in double-stranded form, complicating hybridization assays. When DNA is double-stranded, the sequence information needed by hybridization probes is largely inaccessible due to base pairing within the double helical structure. However, PNA is able to overcome this barrier through a process known as strand invasion. In strand invasion, the non-targeted DNA strand is locally displaced to permit the PNA probe to bind to its target. (See FIG. 4A). (8, 11).

As illustrated in FIG. 4B, a second PNA strand can bind to the PNA-DNA hybrid to yield a PNA-DNA-PNA triple helix. (12-14). In addition, the efficiency of strand invasion can be improved by adding a second PNA to target the displaced strand. (See FIG. 4C). (15). The benefit afforded by strand invasion of PNA in the context of a diagnostic assay is that the target DNA does not have to be denatured to permit hybridization of the probe. Moreover, there is a greater sensitivity to mismatches when targeting duplex versus single-stranded DNA since in the former case, the mismatched probe will be competing with a fully matched duplex rather than an unhybridized single strand. (16, 17).

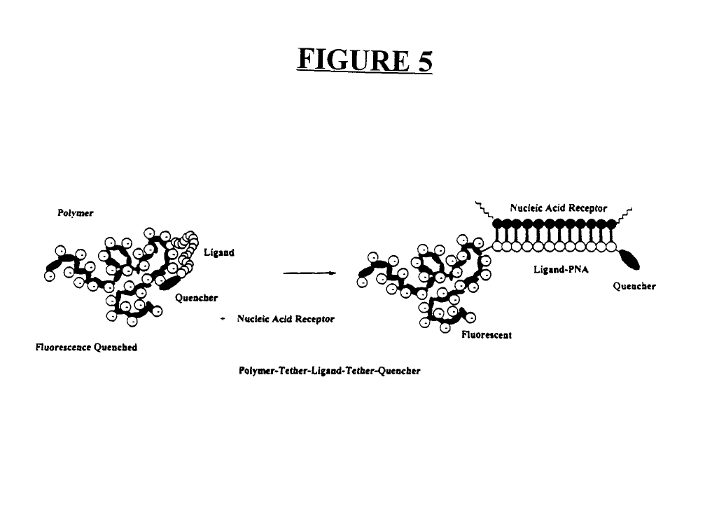

A synthetic PTLT′Q molecule, where L is a specific sequence of PNA, such as a sequence of single stranded DNA or a sequence of single stranded RNA, provides a custom detector for a complementary nucleic acid sequence as illustrated in FIG. 5. In the absence of a complement to the PNA sequence of the PTLT′Q, the moderately hydrophobic PNA sequence is in a tightly coiled configuration in aqueous environments to minimize organic-aqueous interfaces. As a result, the polymer is strongly quenched due to the close proximity of the quencher and polymer. The addition of a receptor (e.g., nucleic acid) that “recognizes” the PNA sequence results in hybridization of the DNA-PNA sequence to produce a structured rigid, double-strand segment that strongly separates the polymer and the quencher and “turns on” the polymer fluorescence. (See illustration of FIG. 5). As discussed above, PNAs can hybridize with complementary strands of DNA and RNA either by direct interaction with a single strand or via strand invasion.

A PNA of approximately 12-20 units is optimal for obtaining a balance of binding specificity and solubility. The PNA can be “inserted” covalently between a fluorescent polymer and quencher by relatively simple synthetic procedures. The complement that is detected may be an oligomer of similar length to the PNA segment or a segment of a larger plasmid.

By developing a suite of PTLT′Q molecules where P is varied, it is possible to have polymers which fluoresce with different “colors” in response to the presence of specific agents. The quencher to be used in this format will most preferably be a species that quenches in a non-radiative fashion and that exhibits a sharp fall-off of quenching efficiency with quencher-polymer separation. The most appropriate quenchers are those that quench the polymer fluorescence by electron transfer or by energy transfer and that form weak ground state charge-transfer complexes. Examples of the polymer for use in the PTLT′Q molecule include, but are not limited to, fluorescent polymers (including conjugated polymers), conjugated polyelectrolytes and dye pendant polymers (including polyelectrolytes). Suitable examples of the fluorescent polymer are conjugated polymers or oligomers including poly phenylene vinylene derivatives, poly (phenyleneethynylene) derivatives, polyphenylene derivatives, polythiophene derivatives, polyfluorene derivatives, neutral, anionic, and cationic conjugated polymers. The dye-pendant polymers can include those which interact through J-aggregation and consequently have very narrow absorption and emission spectra.

Conjugated polymers are extended chromophores whose unit cell or repeat unit may or may not be viewed as a dye. As conjugation (number of repeat units) increases, the absorption and fluorescence spectra shift to longer wavelengths. The shifts maximize after a relatively small number of repeat units (5-6) is attained and show little change thereafter. “QTL” biosensing was first developed with a poly (phenylene vinylene) polyelectrolyte (PPV) and has subsequently been demonstrated with a number of different conjugated polymers. The conjugated polymer used in construction of PTLT′Q molecule may thus be a relatively small oligomer or an extended conjugated polymer chain.

The J-aggregating dye polyelectrolytes offer very attractive possibilities for synthesis of a suite of molecules for multiplexing due to the narrow spectral band of emission in these polymer. Dye polymers that have an ionic fluorescent dye chromophore on each repeat unit on a non-conjugated polymer have previously been shown to exhibit strong J-aggregate absorption and fluorescence. (18-20). Suitable examples of fluorescent dyes for use in the present invention include symmetrical cyanine dye chromophores, unsymmetrical cyanine chromophores, merocyanine dyes, positively charged dye chromophores, negatively charged dye chromophores, and neutral dye chromophores.

These J-aggregate polymers exhibit readily detectable and characteristic absorption and fluorescent transitions. J-aggregate polymers are prepared in a variety of molecular weights ranging from monomer to small oligomer to polymers having a number of polymer repeat units (PRUs) ranging from 6-904. However, in one case the fluorescence emission of the polymer arises from a J-aggregate for polymers having 33 PRUs or higher. The narrow and intense fluorescence of J-aggregates can easily be detected at polymer concentrations (in repeat units) of 10⁻⁹ M. The sensitivity to “superquenching” of these polymers provides a basis for detection of specific target molecules at extremely low levels. Furthermore, the narrow absorption and fluorescence bands of these J-aggregated polymers make it possible to follow changes in the luminescence of several different polymers in the same sample simultaneously.

The PTLT′Q molecule may be employed in solution or supported formats (i.e., the polymer is affixed to a support). Examples of specific supports include a fiber optic, a flexible plastic substrate, porous beads (e.g., organic polymers, natural clays, synthetic clays, particles, membranes, and microporous gels), and solid beads (e.g., organic polymers, silica, natural clays, synthetic clays, and particles). Preferred examples of supported formats include adsorption to a bead, particle, or surface, or a covalent coupling of the entire PTLT′Q moiety to a bead, particle, or surface. In this format, biosensing may be carried out in a static or flow-through mode.

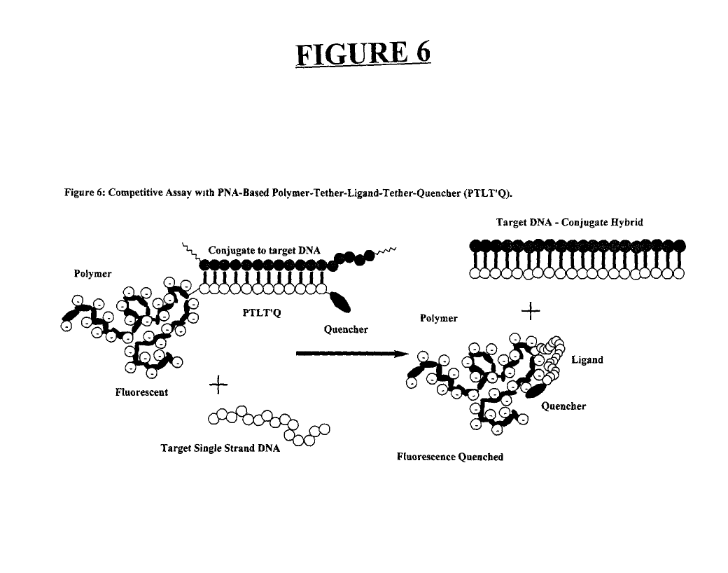

The PTLT′Q molecule may also be employed as a custom competition sensor in a fluorescence quench mode, as can be seen in FIG. 6. In this application, the PTLT′Q molecule is precomplexed to a complement of the target nucleic acid. The complex is chosen to have a longer sequence such that a tighter duplex may be formed with the target DNA than with the PTLT′Q molecule. The presence of the target DNA is sensed following release of the PTLT′Q from its complement as the complement hybridizes with the target DNA. The released PTLT′Q undergoes a quenching of its fluorescence by “collapse” of the dissociated PNA strand.

Having generally described the invention, a further understanding can be obtained by reference to certain specific examples which are provided herein for purposes of illustration only and are not intended to be limiting unless otherwise specified.

### EXAMPLES

### Example 1

A PTLT′Q conjugate can be constructed by connecting a monofunctionalized, negatively charged poly(phenyleneethynylene) (PPE) carboxylate (PT-X) to one terminal of a 12 unit PNA sequence complementary to a target DNA (Y-L-A). Reaction of the resulting PTQ-A fragment with an energy accepting azo dye (AZO) functionalized molecule (B-T′-Q) would result in the formation of a PTLT′Q molecule in which the components are PPE-T-PNA12-T′AZO. The water soluble PTLT′Q would be nonfluorescent due to efficient energy transfer from the PPE to AZO; the AZO would be nonfluorescent. When a solution containing this PTLT′Q molecule is exposed to a target (15 unit) strand of DNA, the resulting hybridization would result in an extension of the PNA strand and a turn-on of fluorescence from the PPE chromophore. The same PTLT′Q molecule may be adsorbed onto cationic (amine-derivatized) polystyrene beads. In this format, the fluorescence of the PPE would be quenched unless hybridization with a complementary strand of DNA occurs. Association of the DNA with the PNA portion of TLT′ would result in an extension of the “connection” between the PPE and AZO and a turn on of the PPE fluorescence.

### Example 2

A PTLT′Q conjugate can be constructed by connecting a cyanine-“capped” poly-L-lysine oligomer of 35 units to a PNA strand of 15 units by amide bond formation with the carboxyl end of the poly-L-lysine segment. The opposite end of the PNA strand can be connected to the same energy accepting azo dye (AZO) and the PTLT′Q functions in the same way. In the absence of the target nucleic acid, the fluorescence of the J-aggregated cyanine poly-L-lysine (P) is quenched by energy transfer to the nonfluorescent AZO; addition of target nucleic acid would result in hybridization and separation of P from AZO. The separation induced by a structured hybrid strand would provide sufficient separation to turn on the fluorescence of P.

### Example 3

The polymer component of the Example 1 is combined with a PNA sequence as described above. This PTL-A segment can be reacted with a functionalized viologen derivative (B-T′-Q) to yield a PTLT′Q in which the quencher is an cationic electron transfer quencher. In the absence of target nucleic acid, the PTLT′Q would be coiled due to hydrophobic interactions and Coulombic attraction of the viologen to the polymer. Addition of the target nucleic acid would result in hybridization and removal of the viologen from the vicinity of the polymer. The removal of the electron transfer quencher would result in a turning on of the polymer fluorescence. Both Example 2 and Example 3 may apply to the PTLT′Q in solution or in the supported format described in Example 1.

The invention of this application is described above both generically, and with regard to specific embodiments. A wide variety of alternatives known to those of ordinary skill in the art can be selected within the generic disclosure, and examples are not be interpreted as limiting, unless specially so indicated. The invention is not otherwise limited, except for the recitation of the claims set forth below. All references cited herein are incorporated in their entirety.

What is claimed is:

1. A chemical moiety comprising a fluorescent polymer bound together by a first tethering element to a recognition element which binds to a target nucleic acid, said recognition element being further bound together by a second tethering element to a property altering element which alters fluorescence emitted by said fluorescent polymer when complexed together to a distinguishable degree, wherein, in the presence of binding of said recognition element to said target nucleic acid, the fluorescence emitted by said fluorescent polymer is altered from that emitted when said binding between said recognition element and said target nucleic acid does not occur.

2. The chemical moiety of claim 1, wherein said recognition element is a sequence of peptide nucleic acids that can recognize and hybridize with said target nucleic acid.

3. The chemical moiety of claim 1, wherein said sequence of peptide nucleic acids is a base sequence complementary to a member selected from the group consisting of a sequence of single stranded DNA and a sequence of single stranded RNA.

4. The chemical moiety of claim 1, wherein said property altering element is selected from the group consisting of methyl viologen, quinones, metal complexes, fluorescent dyes, nonfluorescent dyes and energy accepting, electron accepting and electron donating moieties.

5. The chemical moiety of claim 1, wherein said first and second tethering elements are selected from the group consisting of a single bond, a single divalent atom, a divalent chemical moiety of up to 10 carbon atoms in length and a multivalent chemical moiety.

6. The chemical moiety of claim 1, wherein said fluorescent polymer comprises repeat units including a conjugated backbone.

7. The chemical moiety of claim 6, wherein said fluorescent polymer is selected from a group of conjugated polymers or oligomers consisting of poly phenylene vinylene derivatives, poly (phenyleneethynylene) derivatives, polyphenylene derivatives, polythiophene derivatives, polyfluorine derivatives, neutral, anionic and cationic conjugated polymers.

8. The chemical moiety of claim 1, wherein said fluorescent polymer comprises repeat units each containing a fluorescent dye pendant on a backbone moiety.

9. The chemical moiety of claim 8, wherein the number of repeat units is greater than or equal to 33.

10. The chemical moiety of claim 9, wherein said fluorescent polymer is a J-aggregate.

11. The chemical moiety of claim 8, wherein said fluorescent dye is selected from the group consisting of symmetrical cyanine dye chromophores, unsymmetrical cyanine chromophores, merocyanine dyes, positively charged dye chromophores, negatively charged dye chromophores and neutral dye chromophores.

12. The chemical moiety of claim 1, wherein said fluorescent polymer is affixed to a support.

13. The chemical moiety of claim 12, wherein said support is selected from the group consisting of a fiber optic, a flexible plastic substrate, porous beads solid beads, organic polymers, natural clays, synthetic clays particles, membranes, microporous gels and silica.

14. A method for detecting a target nucleic acid in a sample comprising:
determining the fluorescence emitted by said chemical moiety of claim 1 in the absence of a sample;
adding said chemical moiety to said sample;
permitting said recognition element to bind with target nucleic acid present in said sample;
determining the fluorescence emitted by said fluorescent polymer after said permitting step;
wherein a difference in fluorescence emitted after said permitting step compared with that emitted in the absence of said sample is indicative of the presence of said target nucleic acid.

15. The method of claim 14, wherein the amount of target nucleic acid present in said sample is correlated with the amount of said difference in fluorescence.

16. The method of claim 14, wherein said recognition element is a sequence of peptide nucleic acids that can recognize and hybridize with said target nucleic acid.

17. The method of claim 16, wherein said sequence of peptide nucleic acids is a base sequence complementary to a member selected from the group consisting of a sequence of single stranded DNA and a sequence of single stranded RNA.

18. A method for determining the presence of a target nucleic acid in a sample comprising:
complexing a complement of the target nucleic acid to a chemical moiety of claim 1 to form a PNA:complement nucleic acid complex;
adding said PNA:complement nucleic acid complex to said sample;
permitting said target nucleic acid to compete with said chemical moiety for the binding of said complement; and
determining the fluorescence emitted by said polymer after said permitting step;
wherein the difference in fluorescence emitted after said permitting step compared with that emitted before said permitting step is indicative of the presence of said target nucleic acid.

19. The method of claim 18, wherein the amount of target nucleic acid present in said sample is correlated with the amount of said difference in fluorescence.

20. The method of claim 18, wherein said recognition element is a sequence of peptide nucleic acids that can recognize and hybridize with said target nucleic acid.

21. The method of claim 20, wherein said sequence of peptide nucleic acids is a base sequence complementary to a member selected from the group consisting of a sequence of single stranded DNA and a sequence of single stranded RNA.
